(19) United States
(12) Patent Application Publication
(10) Pub. No.: US 2025/0352041 A1
(43) Pub. Date: Nov. 20, 2025

(54) ELEVATORS FOR MEDICAL DEVICES

(71) Applicant: Boston Scientific Scimed, Inc., Maple Grove, MN (US)

(72) Inventors: Sean POWELL, Holden, MA (US); Diana WALLET, Brighton, MA (US)

(73) Assignee: Boston Scientific Scimed, Inc., Maple Grove, MN (US)

(21) Appl. No.: 19/203,738

(22) Filed: May 9, 2025

Related U.S. Application Data
(60) Provisional application No. 63/647,211, filed on May 14, 2024.

(51) Int. Cl. A61B 1/00; A61B 1/018; A61B 1/273

(52) U.S. Cl. CPC A61B 1/00098; A61B 1/018; A61B 1/273

(57) ABSTRACT

An elevator for a medical device may comprise an axle and a body having: a guide surface, a back surface on an opposite side of the body from the guide surface, a first side surface, and a second side surface. Each of the first side surface and the second side surface may extend between the guide surface and the back surface. The elevator may further comprise a connector extending outward from the first side surface. The connector may include a channel that is configured to receive a control member for pivoting the elevator about the axle.

### CROSS-REFERENCE TO RELATED APPLICATIONS

This application claims the benefit of priority to U.S. Provisional Application No. 63/647,211, filed on May 14, 2024, which is incorporated by reference herein in its entirety.

### TECHNICAL FIELD

Various aspects of this disclosure relate generally to elevators for medical devices. In particular, aspects of this disclosure pertain to an elevator having a connector that decreases an amount of force required to raise the elevator.

### BACKGROUND

Duodenoscopes may include a handle and a shaft, and the shaft may be insertable into a body lumen of a subject. The shaft may terminate in a distal tip portion, which may include features such as optical elements (e.g., camera, lighting), air/water outlets, and working channel openings. An elevator may be disposed at a distal tip and may be actuatable in order to change an orientation of an accessory device passed through the working channel. For example, the elevator may be pivotable or otherwise movable. During a procedure, different accessory devices may be inserted and/or removed from the working channel of the duodenoscope. Therefore, a need exists for elevators of medical devices that facilitate insertion and removal of various accessory devices.

### SUMMARY

Each of the aspects disclosed herein may include one or more of the features described in connection with any of the other disclosed aspects. Aspects of the disclosure may relate to duodenoscope elevators having connection points for control wires that decrease or reduce a force required to actuate the elevators.

An elevator for a medical device may comprise an axle and a body having: a guide surface, a back surface on an opposite side of the body from the guide surface, a first side surface, and a second side surface. Each of the first side surface and the second side surface may extend between the guide surface and the back surface. The elevator may further comprise a connector extending outward from the first side surface. The connector may include a channel that is configured to receive a control member for pivoting the elevator about the axle.

Any of the aspects disclosed herein may have any of the following features. The connector may include (a) a first portion that extends outwardly from the first side surface and (b) a second portion that extends outwardly from the back surface. The channel may extend through the first portion and the second portion. The second portion may be configured to be fixedly coupled to an end of the control member. The channel may be configured to increase a moment arm of the control member. The moment arm of the control member may be greater than or equal to 150% of a hypothetical moment arm of a hypothetical control member that extends only through a portion of the channel extending through the second portion. A first portion of the channel extending through the first portion may be approximately perpendicular to a second portion of the channel extending through the second portion. Each of the first portion of the channel and the second portion of the channel may be approximately perpendicular to a longitudinal axis of the elevator. The channel may have an opening on an end of the channel. At least a portion of the channel may have an open side. A first portion of the channel may have an open distal side. A second portion of the channel may have an open lateral side. The first portion of the channel may be closer to the guide surface than the second portion of the channel is. The open distal side may be continuous with the open lateral side. The open distal side may be continuous with the opening on the end of the channel. The connector may extend upwardly beyond the guide surface. A first end of the connector is above the guide surface. A second end of the connector may be adjacent the second side surface.

In another aspect, an elevator for a medical device may comprise: an axle and a body having: a guide surface, a back surface on an opposite side of the body from the guide surface, a first side surface, and a connector configured to couple a control member for pivoting the elevator about the axle to the body. The connector may include (a) a first portion extending along the first side surface and (b) a second portion extending along the back surface.

Any of the aspects disclosed herein may have any of the following features, alone or in any combination. The connector may be configured such that the control member extends through the first portion and the second portion. A channel may extend through the first portion and the second portion. The channel may have at least one open side.

In a further aspect, a medical device may comprise: a shaft, a control member extending through the shaft, and a distal tip, including an elevator. The elevator may have a guide surface, a back surface on an opposite side of the elevator from the guide surface, a first side surface extending between the guide surface and the back surface, and a connector continuous with the first side surface. The connector may include a channel that receives the control member.

Any of the aspects disclosed herein may have any of the following features, alone or in any combination. The connector may extend along the back surface. The channel may extend along the first side surface and the back surface.

Any of the features above may be combined in any suitable combination or used separately.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2C depict an elevator of the exemplary medical device.

FIGS. 3A-3D depict a distal tip of the medical device having the elevator of FIGS. 2A-2C, with the elevator in various configurations.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. Some Figures include arrows labeled “P” and “D” to indicate proximal and distal directions, respectively. The term “exemplary” is used in the sense of “example,” rather than “ideal.” The term “approximately,” or like terms (e.g., “substantially”), includes values+/−10% of a stated value.

Medical devices, such as duodenoscopes, may include elevators that are used to, for example, change an orientation of one or more accessory medical devices. An elevator may be actuated by an actuator (e.g., a lever) on a handle of a medical device. The actuator may be coupled to a control member (e.g., a wire or cable), which may, in turn, be coupled to the elevator. Thus, actuating the actuator may cause the elevator to raise and/or lower. It may be desirable to decrease an amount of force that is required in order to actuate the actuator. Thus, the disclosed aspects include an elevator with a control member connector that increases a lever arm (moment arm) of the elevator, thereby decreasing an amount of force required to raise and/or lower the elevator. The connector may include a channel through which the control member extends, thus displacing the control member and helping to create a larger lever arm. Aspects of the invention may facilitate use of elevators with smaller lengths (along a longitudinal axis of the elevator). For example, aspects of the invention may offset an otherwise decreased lever length of a smaller elevator, due to a smaller distance between an axle of the elevator and a portion of a body of the elevator to which the control member is coupled.

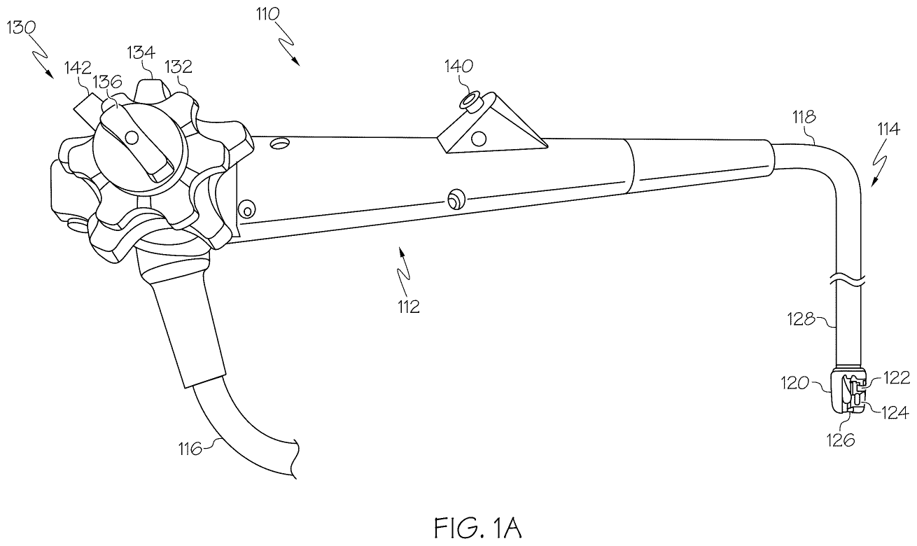

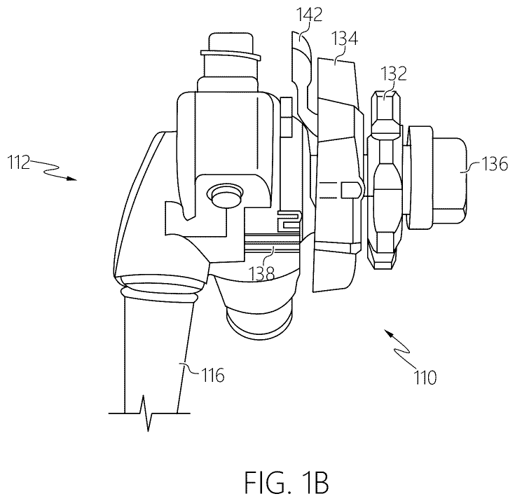

FIG. 1A depicts an exemplary medical device 110 (e.g., a duodenoscope or other type of scope, such as an endoscopic ultrasound (EUS) scope) having a handle 112 and an insertion portion 114. FIG. 1B shows a proximal end of handle 112. Medical device 110 may also include an umbilicus 116 for purposes of connecting medical device 110 to sources of, e.g., air, water, suction, power, etc., as well as to image processing and/or viewing equipment. Insertion portion 114 may include a sheath or shaft 118 and a distal tip 120. Distal tip 120 may include an imaging device 122 (e.g., a camera) and a lighting source 124 (e.g., an LED or an optical fiber). In some examples, as shown in FIG. 1A, distal tip 120 may be side-facing (i.e., side viewing). That is, imaging device 122 and lighting source 124 may face radially outward, perpendicularly, approximately perpendicularly, or otherwise transverse to a longitudinal axis of shaft 118 and distal tip 120.

Distal tip 120 may also include an elevator 126 for changing an orientation of a medical instrument inserted in a working channel of medical device 110. Elevator 126 may alternatively be referred to as a swing stand, pivot stand, raising base, or any suitable other term. Elevator 126 may be rotatable via, e.g., an actuation wire or another control element that extends from handle 112, through shaft 118, to elevator 126.

A distal portion of shaft 118 that is connected to distal tip 120 may have a steerable section 128. Steerable section 128 may include, e.g., an articulation joint (e.g., articulation region). Shaft 118 and steerable section 128 may include a variety of structures which are known or may become known in the art.

Handle 112 may have one or more actuators/control mechanisms 130. One or more of control mechanisms 130 may provide control over steerable section 128. One or more of control mechanisms may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include a first locking mechanism 136 and a second locking mechanism 142 (e.g., knobs or levers) for preventing steering and/or braking of steerable section 128 in at least one of an up, down, left, or right direction. Handle 112 may include a control lever 138 (see FIG. 1B) or other actuator. Control lever 138 may raise and/or lower elevator 126, via one or more connections between control lever 138 and an actuating wire that extends from control lever 138, through shaft 118, to elevator 126. A port 140 may allow passage of a medical instrument through port 140, into a working channel of the medical device 110, through shaft 118, to distal tip 120.

Although FIG. 1A depicts a side-viewing medical device 110 (a device having imaging device 122 and/or lighting source 124 facing substantially radially outward), it will be appreciated that medical device 110 may alternatively be a forward-viewing device (a device having imaging device 122 and/or lighting source 124 facing substantially distally). In aspects, medical device 110 may include both a side-viewing imaging device 122 and/or lighting source 124 and a forward-facing imaging device 122 and/or lighting source 124.

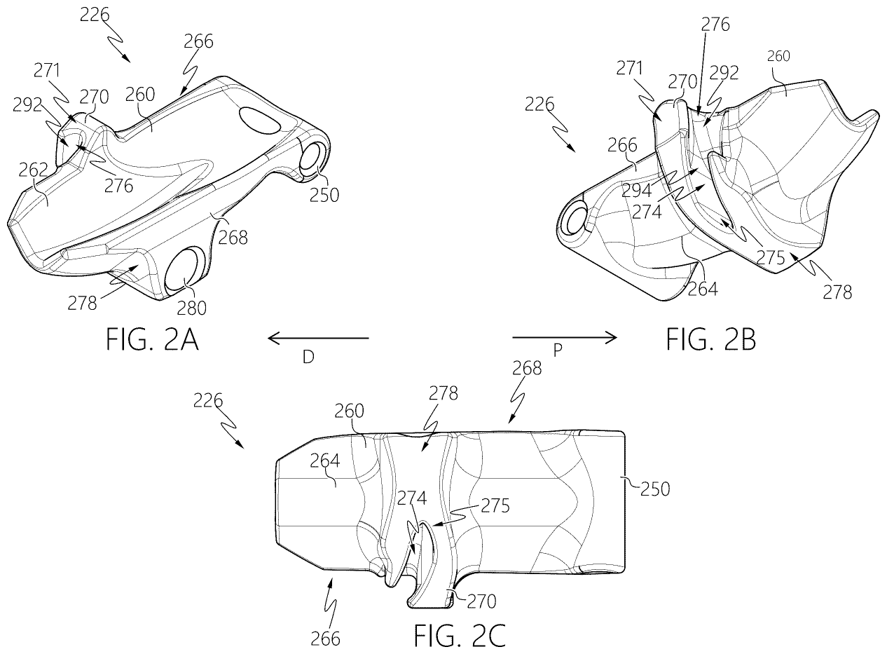

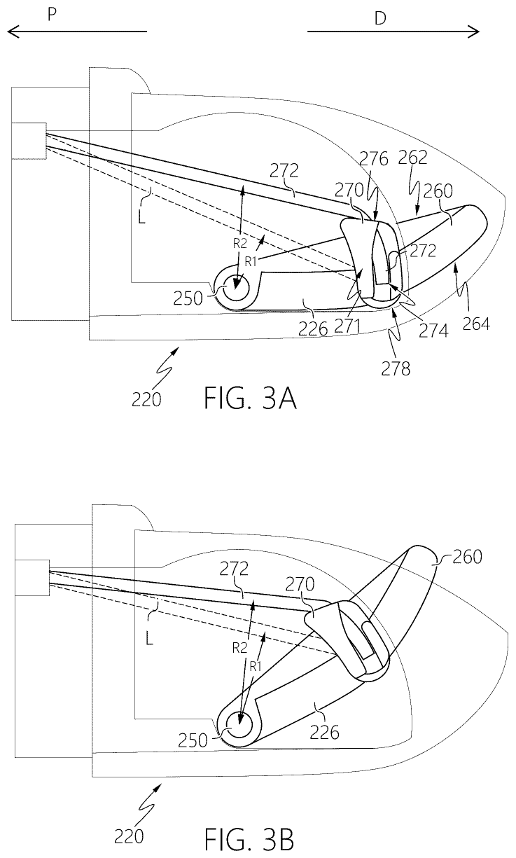

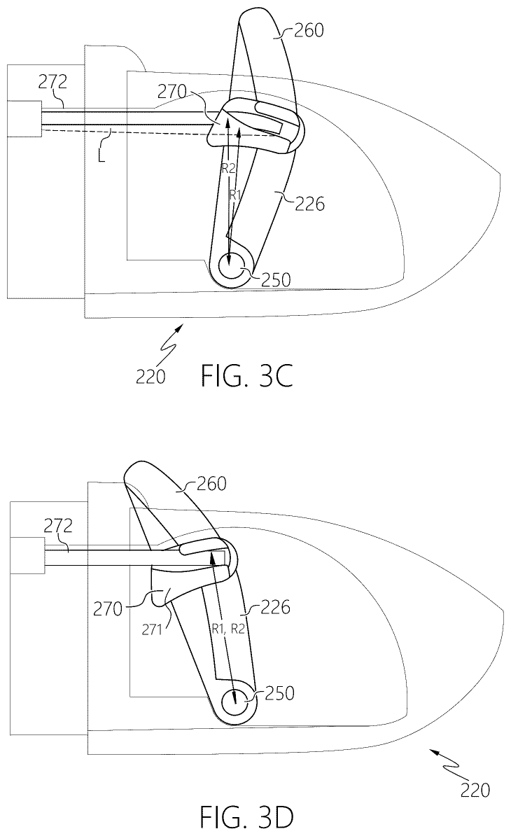

FIGS. 2A-2C depict features of an elevator 226, which may have any of the features of elevator 126 and may be used in conjunction with medical device 100. FIG. 2A shows a top perspective view of elevator 226, FIG. 2B shows a bottom/back perspective view of elevator 226, and FIG. 2C shows a bottom/back plan view of elevator 226. FIGS. 3A-3D show a distal tip 220, having any of the features of distal tip 120, with elevator 226 in various configurations. In FIGS. 3A-3D, portions of distal tip 220 are shown as transparent, in order to illustrate features of elevator 226. Distal tip 220 may be used in conjunction with medical device 100.

Elevator 226 may have a proximal axle 250 and a body 260 extending distally from axle 250. Body 260 may have a guide surface 262 (FIG. 2A), which may be a front surface of body 260. Guide surface 262 may receive and contact a portion of an accessory device (e.g., an endoscopic instrument, not shown) that is extended through a working channel of medical device 100. Elevator 226 may rotate or pivot about axle 250 in order to change an orientation of the accessory device. For example, a first end of a control member 272 (e.g., a wire or a cable, shown in FIGS. 3A-3D) may be coupled (e.g., removably coupled) to elevator 226 by a connector 270. Although not shown, a second end of control member 272 may be coupled to (e.g., removably coupled) control lever 138 (FIG. 1B). Aspects of elevator 226, including connector 270, are described in further detail below.

As mentioned above, elevator 226 may have axle 250. Axle 250 may have any of the features of any axle known in the art to be used with a medical device elevator. For example, axle 250 may have a substantially cylindrical shape. Axle 250 may be received within a corresponding portion or portions of distal tip 220, such that elevator 226 is rotatable or pivotable about axle 250.

Body 260 may have guide surface 262 on one side of body 260. Guide surface 262 may be sized and shaped so as to receive, contact, and adjust the orientation of an accessory device extended through a working channel of medical device 100. Guide surface 262 may face a same direction as imaging device 122 (FIG. 1A) when elevator 226 is in a lowered configuration and may face proximally when elevator 226 is in a raised configuration. On an opposite side from guide surface 262, body 260 may have a back surface 264. Back surface 264 may face a housing of distal tip 220 when elevator 226 is in a lowered configuration and may face distally when elevator 226 is in the raised configuration.

Body 260 may also have a first side surface 266 and a second side surface 268. First side surface 266 and second side surface 268 may be on opposite sides of body 260. Each of first side surface 266 and second side surface 268 may extend between guide surface 262 and back surface 264. Second side surface 268 may be closer to imaging device 122 and lighting source(s) 124.

Connector 270 extend outwardly from first side surface 266 and back surface 264. Connector 270 may be a portion of body 260. Alternatively, at least portions of connector 270 may be flush with first side surface 266 and/or back surface 264. Connector 270 may have a first portion 271, which extends from and along first side surface 266, and a second portion 278, which extends along back surface 264. In some examples, first portion 271 may extend upwardly past guide surface 262 (above guide surface 262 in the views of FIGS. 2A and 3A). On other examples, first portion 271 may have an end that is level with a portion of guide surface 262 that intersects first side surface 266 (not extend above guide surface 262). Each of first portion 271 and second portion 278 may extend generally perpendicularly to a longitudinal axis of elevator 226 (a longitudinal axis of elevator 226 may extend parallel to the proximal/distal directions labeled in FIG. 2C, from axle 250 to a distal surface of elevator 226). First portion 271 and second portion 278 may extend approximately perpendicularly to one another.

In examples, connector 270 may be directly coupled to first side surface 266 and/or back surface 264. For example, no gap may be disposed laterally between first side surface 266 and connector 270. A continuous surface may be formed between adjacent portions of first side surface 266 and connector 270. Similarly, a continuous surface may be formed between adjacent portions of back surface 264 and connector 270. Elevator 226 may thus lack a separate control arm that extends from axle 250 and/or a proximal portion of body 260.

Connector 270 may be configured to couple control member 272 to elevator 226. For example, connector 270 may define a channel 274. Channel 274 may extend through connector 270, through first portion 271 and second portion 278. For example, channel 274 may have an opening 276 at an end of channel 274 that is at or near an intersection between guide surface 262 and first side surface 266. Opening 276 may be at a top of channel 274 (and a top of first portion 271) in the frame of reference of FIGS. 2A and 2B. A portion of channel 274 extending through first portion 271 may extend approximately perpendicularly to a portion of channel 274 extending through second portion 278. The portions of channel 274 extending through first portion 271 and second portion 278 may each be approximately perpendicular to a longitudinal axis of elevator 226 (the longitudinal axis of elevator 226 may be parallel to the proximal/distal directions, indicated in FIG. 2C).

Along at least a portion of first portion 271 (e.g., along an entirety of first portion 271), channel 274 may have an open side, such that channel 274 is exposed along first portion 271. The open side may be continuous with opening 276 or may be discrete from opening 276. The open side of channel 274 may be a radially outer side of channel 274, relative to a longitudinal axis of channel 274. The open side of channel 274 may assist in affixing control member 272 within channel 274 and/or in allowing control member 272 to move as necessary within channel 274.

As best shown in FIGS. 3A-3D, near opening 276, a distal side of a first portion of channel 274 may be a distal open side 292, and a laterally outer side surface of channel 274 may be enclosed. Further toward back surface 264, an outer side surface of a second portion of channel 274 may include an open lateral side 294, and a distal side of channel 274 may be enclosed. Distal open side 292 (and the first portion of channel 274) may be closer to guide surface 262 than lateral open side 294 (and the second portion of channel 274) is. However, such an arrangement is merely exemplary and alternative portions may be open. In some aspects, open lateral side 294 may be continuous with open distal side 292. Alternatively, open lateral side 294 may be discrete from open distal side 292. In examples, open distal side 292 may be continuous with opening 276.

An arrangement of open sides of channel 274 may assist with retaining control member 272 within channel 274 and/or inhibiting control member 272 from slipping out of channel 274 along an entire range of motion of elevator 226. For example, a proximal surface of channel 274 may be closed along an entirety of channel 274 (or at least along a portion of channel 274 having open distal side 292) to inhibit control member 272 from slipping proximally out of channel 274 as elevator 226 raises/lowers.

Along at least a portion of second portion 278, channel 274 may lack an open side and may have fully enclosed sides. The portion of channel 274 that has an open side may terminate at an end 275, which may be on first portion 271 or second portion 278 of connector 270. At an end of second portion 278 that is farthest from first side surface 266 (e.g., at an end of second portion 278 that is at or near (e.g., adjacent to) second side surface 268), an end of channel 274 may have an opening 280. Opening 280 and/or a portion of channel 274 that extends through second portion 278 may serve as a ferrule or other mechanism for securing control member 272 within channel 274. For example, an end of control member 272 may be fixedly coupled within channel 274 of second portion 278 by a crimp, adhesive, friction fit, or any other suitable mechanism. Second portion 278 may be configured to be fixedly coupled to an end control member 272 (e.g., via opening 280). In alternative aspects, second portion 278 may extend along only a portion of back surface 264, and/or channel 274 may extend along only a portion of second portion 278.

In examples, second portion 278 (e.g., opening 280) may be a connection point/location for control member 272. A portion of channel 274 extending through first portion 271 may displace control member 272 from the connection point (e.g., raising control member 272 about the connection point, in a frame of reference of FIGS. 2A and 3A). In other words, control member 272 may be offset from a connection point where control member 272 is fixedly coupled to elevator 228 by first portion 271.

Aspects of connector 270 are merely exemplary. In alternatives, connector 270 may lack second portion 278 and may only include first portion 271 extending from first side surface 266 and/or channel 274 may not extend through second portion 278. In such aspects, control member 272 may be secured within first portion 271 of connector 270 (e.g., by a ferrule, adhesive, etc.). In further aspects, first portion 271 may terminate above (in the frame of reference of FIGS. 2A and 3A) back surface 264. In further alternatives, an entirety of channel 274 may have an open side. In another aspect, channel 274 may be enclosed along its entire length (except for openings 276 and 280 at either end of channel 274).

In another alternative, first portion 271 may extend longitudinally along first side surface 266 (e.g., approximately parallel to a longitudinal axis of elevator 226). First portion 271 may include a channel that extends through first portion (e.g., approximately parallel to the longitudinal axis of elevator 226). The channel may be approximately at a same height (in the up/down direction of FIGS. 2A and 3A) as guide surface 262, above guide surface 262, or below guide surface 262 (but above back surface 264). In such an alternative, second portion 278 may be omitted or be included. The alternatives above are merely exemplary, and aspects of the alternatives may be combined in any suitable combination.

FIGS. 3A-3D illustrate how connector 270 may help to decrease a force required to pivot or rotate elevator 226 (e.g., using control lever 138). The broken lines in FIGS. 3A-3D illustrate a theoretical control member L that is coupled to elevator 226 at or near back surface 264 of body 260. For example, control member L illustrates a hypothetical control member positioning if connector 270 lacked first portion 271 and included only a portion extending along back surface 264 (e.g., second portion 278). The distance R1 denotes a lever arm distance for control member L (a distance between axle 250 and control member L). The distance R2 denotes a lever arm distance for control member 272, which extends through channel 274 of connector 270 (a distance between axle 250 and control member 272). In aspects, R1 may be a radius of a circle along which control wire L moves, and R2 may be a radius of a circle along which control member 272 moves. R1 and R2 are shown schematically and are not intended to depict exact relative distances/exact positions of lever arms. Instead, R1 and R2 are intended to depict relative differences in length of the respective lever arms.

FIG. 3A shows elevator 226 in a fully lowered configuration within distal tip 220. As shown in FIG. 3A, control member 272 extends through channel 274 and out of opening 276. As shown in FIG. 3A, R2 is larger than R1. In some examples, R2 may be greater than or equal to 150% of R1. Because control member 272 extends through channel 274 along first portion 271 of connector 270, a portion of control member 272 that extends proximally from opening 276 is separated or spaced away further from axle 250 than hypothetical control member L extending from a portion of elevator 226 near back surface 264. This greater length of R2 mean that less force is required to be exerted on control member 272 by control lever 138 in order to exert equal torque on elevator 226 to rotate/pivot elevator 226.

FIGS. 3B-3D show elevator 226 is progressively more raised configurations. As shown in FIG. 3B, as elevator 226 begins to raise, R2 continues to be larger than R1. As discussed above, a greater value of R2 means that less force is required to be exerted in order to obtain the same amount of torque on elevator 226. However, a relative difference between R2 and R1 may be smaller, because of a changing angle of elevator 226 and control member 272. In FIG. 3C, as elevator 226 continues to raise, positions of control member 272 and hypothetical control member L may begin to converge, such that R2 is only slightly larger than R1.

In FIG. 3D, when elevator 226 is fully raised, control member 272 may fully merge with hypothetical control member L, such that hypothetical control member L is not shown in FIG. 3D. For example, first portion 271 of connector 270 may extend approximately parallel to a central longitudinal axis of distal tip 220 (which may be parallel to the proximal/distal directions), such that control member 272 may extend approximately parallel to the central longitudinal axis of distal tip 220. Thus, control member 272 may follow a same or similar path to a hypothetical control member L coupled near back surface 264 of elevator 226. As such, R1 and R2 may be the same or approximately the same.

Accordingly, connector 270 helps to increase a radius along with control member 272 travels. This increased radius (due to control member 272 effectively being displaced so as to be level with or above an edge of guide surface 262) before extending proximally decreases an amount of force required to be exerted by control lever 138. Furthermore, connector 270 acts as a spool from which control member 272 unwinds as elevator 226 raises, as shown in FIGS. 3A-3D. Thus, control member 272 does not move from a fixed point. Therefore, regardless of a position of elevator 226, lever arm R2 is optimized (i.e., lever arm R2 is optimized along an entire scope of movement of elevator 226). Any of the aspects disclosed herein may increase a radius along which control member 272 travels by displacing control member 272 from the point at which control member 272 is coupled to elevator 226 (e.g., along back surface 264 of elevator 226).

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented embodiments can be combined to achieve a same or similar result as one or more of the disclosed embodiments. Accordingly, the invention is not to be considered as limited by the foregoing description.

We claim:

1. An elevator for a medical device, the elevator comprising:
an axle; and
a body having:
a guide surface;
a back surface on an opposite side of the body from the guide surface;
a first side surface;
a second side surface, wherein each of the first side surface and the second side surface extends between the guide surface and the back surface; and
a connector extending outward from the first side surface, wherein the connector includes a channel that is configured to receive a control member for pivoting the elevator about the axle.

2. The elevator of claim 1, wherein the connector includes (a) a first portion that extends outwardly from the first side surface and (b) a second portion that extends outwardly from the back surface.

3. The elevator of claim 2, wherein the channel extends through the first portion and the second portion.

4. The elevator of claim 3, wherein the second portion is configured to be fixedly coupled to an end of the control member.

5. The elevator of claim 4, wherein the channel is configured to increase a moment arm of the control member.

6. The elevator of claim 5, wherein the moment arm of the control member greater than or equal to 150% of a hypothetical moment arm of a hypothetical control member that extends only through a portion of the channel extending through the second portion.

7. The elevator of claim 2, where a first portion of the channel extending through the first portion is approximately perpendicular to a second portion of the channel extending through the second portion.

8. The elevator of claim 7, wherein each of the first portion of the channel and the second portion of the channel is approximately perpendicular to a longitudinal axis of the elevator.

9. The elevator of claim 1, wherein the channel has an opening on an end of the channel, and wherein at least a portion of the channel has an open side.

10. The elevator of claim 9, wherein a first portion of the channel has an open distal side, and wherein a second portion of the channel has an open lateral side.

11. The elevator of claim 10, wherein the first portion of the channel is closer to the guide surface than the second portion of the channel is.

12. The elevator of claim 10, wherein the open distal side is continuous with the open lateral side.

13. The elevator of claim 12, wherein the open distal side is continuous with the opening on the end of the channel.

14. The elevator of claim 1, wherein the connector extends upwardly beyond the guide surface.

15. The elevator of claim 14, wherein a first end of the connector is above the guide surface, and wherein a second end of the connector is adjacent the second side surface.

16. An elevator for a medical device, the elevator comprising:
an axle; and
a body having:
a guide surface;
a back surface on an opposite side of the body from the guide surface;
a first side surface; and
a connector configured to couple a control member for pivoting the elevator about the axle to the body, wherein the connector includes (a) a first portion extending along the first side surface and (b) a second portion extending along the back surface.

17. The elevator of claim 16, wherein the connector is configured such that the control member extends through the first portion and the second portion.

18. The elevator of claim 16, wherein a channel extends through the first portion and the second portion, and wherein the channel has at least one open side.

19. A medical device comprising:
a shaft;
a control member extending through the shaft; and
a distal tip, including an elevator having:
a guide surface;
a back surface on an opposite side of the elevator from the guide surface;
a first side surface extending between the guide surface and the back surface; and
a connector continuous with the first side surface, wherein the connector includes a channel that receives the control member.

20. The medical device of claim 19, wherein the connector extends along the back surface, and wherein the channel extends along the first side surface and the back surface.
